(19) United States
(12) Patent Application Publication
(10) Pub. No.: US 2020/0046876 A1
(43) Pub. Date: Feb. 13, 2020

(54) HYDROGEL SURGICAL DRESSING PRODUCT HAVING A MULTI-DIMENSIONAL FLEXIBLE HYDROPHILIC STRUCTURE-LINKAGE COMPOSITE

(71) Applicant: Chuang Sheng Medicine Equipment Co. Ltd., Taoyuan City (TW)

(72) Inventor: Chih Chung LIU, Taoyuan City (TW)

(21) Appl. No.: 16/436,384

(22) Filed: Jun. 10, 2019

(30) Foreign Application Priority Data
Aug. 13, 2018 (MY) PI 2018702844

(51) Int. Cl. A61L 26/00; A61F 13/02

(52) U.S. Cl. CPC A61L 26/008; A61L 15/60; A61F 13/0213

(57) ABSTRACT

This invention provides a hydrogel surgical dressing product having a designed multi-dimensional flexible hydrophilic structure-linkage composite, comprising: a hydrogel cushion layer, and a multiple of hydrophilic microsphere, wherein said multi-dimensional flexible hydrophilic structure-linkage composite comprising a hydrophilic multi-dimensional flexible structure-linkage membrane, and a multiple of structure-linkage holes positioned into said hydrophilic multi-dimensional flexible structure-linkage membrane, and the sizes of structure-linkage holes changed with the degree of compelling force, resulting in a multi-dimensional flexible structure-linkage relationship and a consolidating structure.

### FIELD OF INVENTION

This invention provides a hydrogel surgical dressing product having a designed multi-dimensional flexible hydrophilic structure-linkage composite. Particularly, the hydrogel surgical dressing product could be applied in wound management, tissue repairing, cosmetic and plastic surgery, medical cosmetology, and specific clinical applications.

### BACKGROUND OF THE INVENTION

The gel sheet of the present invention obtained as above can be used for a skin care product, a cosmetic base material, a beauty base material, a clean product, an external preparation, a base material for pharmaceutical products, a quasi-drug, a wound dressing, an antiadhesive film, a drug delivery system, a cell culture base material, a regenerative medicine base material, an air freshener, a deodorant, an insect repellent, an insecticide, a base material for agrochemicals, a base material for diagnosis agents, a solving material for chemical reaction or enzyme reaction, a base material for chemical sensors, a base material for biosensors, food, and other purposes and is particularly preferably expected to be applied as wound dressings, such as a wound dressing sheet.

In the clinical application, the tissue defect is often repaired, but the autologous transplantation has a limited source, while Allogeneic and xenotransplantation have high risk of infection. Therefore, nowadays more and more different organic, inorganic and metallic materials are used in tissue engineering. In addition, in order to avoid two operations, the use of biodegradable materials is necessary. At present, the use of different materials has their advantages and disadvantages, in order to solve different medical problems, the development of new functional composite materials is still one of the focus of research. Tissue engineering is an interdisciplinary field linking engineering and biology. Tissue engineering develops biological substrates that can repair, restore or improve the function of the tissue. The tissue engineering involved three main strategies: using in vitro cells or cell substitutes to replace limited tissue function, inducing tissue formation, such as growth factor (growth factors), and developing biological scaffolds (scaffold) in favor of tissue repairing and regeneration. Therefore, the key factor in the development of scaffolds is to mimic the physical and biological functions of the extracellular matrix (ECM) as a growth environment, which is an important development technique in cell culture substrates. In minimally invasive surgery, it is necessary to develop different functional materials for different types of clinical needs in different ways, but the thinning or micronized biomedical materials have many bottlenecks to break through, further making the thin or micronized biomedical materials more active and more limited.

Currently, hydrogel dressings and related structures, although there are many patented designs, but due to the lack of experience and poor design of material science and molecular structure design, and due to limited experience in surface science and chemical structural characteristics of chemicals limited experience, there is still no good solutions after years of testing. At present, through the acquisition of know-how or patented technology of the medical market, still have off gum, degumming and even often caused by gel-layer blocks or the collapse, falling gum and other problems after water absorption and swelling. For example, Taiwan patent application 101,150,848 titled “Skin Moisturizing Film” mainly includes: a substrate, the system is made up of the film of PU material, which causes the base to have the function of waterproof, transparent and breathable; a support layer, the upper side of which is arranged on one surface of the base; an exudate absorption layer is arranged on one surface of the support layer, the leachate absorption layer is attached directly to the skin, and the leachate absorption layer is composed of a hydrogel. Similarly, a wound dressing as shown in Taiwan patent M419555, includes: a base layer made of polyurethane (polyurethanes), a medical pressure adhesive layer is adhered to the base layer, and the top of which is defined as an edge region and a body region; a support layer made of elastic and ductile materials. It is adhered to the body region of the medical pressure adhesive layer as described above, and an exudate absorption layer is composed of hydrogel and is combined with the support layer for direct contact with the wound. Additionally, a structure of an anti-scar means of Taiwan patent M531256, comprising a supporting layer having a first surface and a second surface, was provided. Said supporting layer is prepared from a mono-dimension flexible and expend fiber. The anti-scar means comprise a surface layer positioned on said first surface and a polymeric pressure-sensitive adhesion layer positioned on said second surface. Said polymeric pressure-sensitive adhesion layer could be prepared from carboxymethyl cellulose, styrene-butadiene rubber (SBR), polyethylene pyrrolidone and poly (ethylene glycol).

The key skill in prior art is always to use an adhesive technology to prepare the target product. However, there are several problems, such as a loose conformation, a collapsed structure, and a falling off situations after swelling in clinical applications, which must be solved. Hence, a good hydrogel dressing is difficult to be developed and obtained. For solving the falling off and weak adhering problems, Taiwan patent I504420 provides a hydrogel dressing, which comprises a complex sheet consist of a membrane layer and a cloth layer coated with un-curing hydrogel. The un-curing hydrogel could be cured by UV light to form a hydrogel layer. However, the coating step is difficult to be applied to the system of a hydrogel and a cloth layer. It is because there is always a surface tension and a surface energy barrier on the cloth layer, which will form a surface barrier and preferably prevent from forming a highly viscous coating solution, for example, an uncurable hydrogel solution. A stable adhesive and laminating structure in a hydrogel dressing is difficult to be obtained. Furthermore, Taiwan patent I5437551 provides a dressing, which comprises an inner layer and an outer layer, wherein said inner layer consists of a hydrogel layer and a melt-blown non-woven layer. Said outer layer consists of a hydrogel layer and a melt-blown non-woven layer. The outer layer comprises a polyurethane membrane and a pressure-sensing adhesive layer, wherein the melt-blown nonwoven layer and the hydrogel layer are coated with ultraviolet light to form a network polymer layer between the melt-blown nonwoven layer and the hydrogel layer, and the fiber exposed in part of the melt-blown nonwoven layer is stabilized with that of the outer layer; wherein the dressing must be made of the hydrogel layer, the network polymer layer, the melt-blown nonwoven layer, the pressure adhesive layer and the polyurethane film in order. In prior art, but also for the structure of painstaking effort, but still unable to avoid the limitation of the adhesion and adhesive technology, in the non-woven film and hydrogels, in the hope that through the exposure of fiber and pressure adhesive, or with hydrogels to produce mesh staggered polymer layer, in an attempt to strengthen the structure, though, it still ignores the structural disintegration and delamination caused by the swelling. Such a design and Taiwan patent I504420 has a very high technical similarity, and therefore, has not been able to stabilize the glue, preventing collapse of the product.

In view of the reasons above, the applicant has engaged in biomedical engineering, materials, interfaces and molecular structure research. Through continuous study and related research, a structural dynamic characteristic of a multi-dimensional flexible hydrogel dressing, in particular, a multi-dimensional flexible structure-linkage film and a comprehensive gel dressing has been created. In the clinical applications, the products of prevent inventions can be satisfied with the clinical treatment and care of wounds with high activity focus and joint.

### SUMMARY OF THE PRESENT INVENTION

This invention provides a designed multi-dimensional flexible hydrophilic structure-linkage module in a hydrogel surgical dressing product to solve the clinic problems of traditional hydrogel dressings, in particular, cracking and collapsed structures being happened after swelling with water or tissue fluid in medical applications. The designed module provides a structure-linkage of each part of the hydrogel surgical dressing product instead of the traditional design of multiple adhering layers. Therefore, the problems observed in prior art could be overcome.

This invention provides a designed multi-dimensional flexible hydrophilic structure-linkage module in a hydrogel surgical dressing product. The multi-dimensional flexible hydrophilic structure-linkage module could be achieved through a kind of a multi-dimensional flexible hydrophilic structure-linkage composite.

Particularly, a multi-dimensional flexible structure-linkage relationship could be achieved through a designed structure-linkage holes positioned into said hydrophilic multi-dimensional flexible structure-linkage membrane, and the sizes of structure-linkage holes changed with the degree of compelling force, resulting in a multi-dimensional flexible structure-linkage relationship.

That is, the invention provides a hydrogel surgical dressing product having a multi-dimensional flexible hydrophilic structure-linkage composite, comprising: a hydrogel cushion layer, and a multi-dimensional flexible hydrophilic structure-linkage composite, wherein said multi-dimensional flexible hydrophilic structure-linkage composite comprising a hydrophilic multi-dimensional flexible structure-linkage membrane, and a multiple of structure-linkage holes positioned into said hydrophilic multi-dimensional flexible structure-linkage membrane, and the sizes of structure-linkage holes change with the degree of compelling force, resulting in a multi-dimensional flexible structure-linkage relationship, characterized in that said hydrogel cushion layer comprising a multiple of hydrophilic structure-linkage fibers and a multiple of hydrogel micro balls, said hydrophilic structure-linkage fiber adsorbs side by side with said hydrogel micro ball in said hydrogel cushion layer; and said hydrogel cushion layer is supported by a hydrophilic structure-linkage network forming through a network connection among said hydrophilic structure-linkage fibers and said hydrophilic structure-linkage fibers, wherein said network connection comprising a first partial connection occurring into said structure-linkage holes, a second partial connection surrounding said structure-linkage holes, and a third connection linking said first partial connection and said second partial connection to form said structure-linkage multi-dimensional flexible structure-linkage relationship; and a multiple of hydrophilic microsphere is positioned in said hydrogel cushion layer, wherein said hydrophilic microsphere adsorbs side by side with said hydrogel micro ball, and adsorbs side by side with said hydrophilic structure-linkage fiber, resulting in a consolidated structure of said hydrogel cushion layer.

Preferably, said hydrophilic structure-linkage fiber comprising a material selected from a group of polymethacrylate, polymethacrylate derivative, polysiloxane derivative, carboxymethylcellulose, alginate, polycarbohydrate, the hyaluronic acid, collagen, the vegetative protein, gelatin, polylactides, polyethylene pyrrole, polyethylene glycol, and thereof.

Preferably, said hydrogel micro ball comprising a material selected from a group of polymethacrylate, polymethacrylate derivative, polysiloxane derivative, carboxymethylcellulose, alginate, polycarbohydrate, hyaluronic acid, collagen, the vegetative protein, gelatin, polylactides, polyethylene pyrrole, polyethylene glycol, and thereof.

Preferably, said hydrophilic microsphere comprising a material selected from a group of aminoacid, glucosamine, water, oxygen, ethyl alcohol, propyl alcohol, glycerine, small molecular water-soluble polymethyl acrylic acid derivative, small molecular polysiloxane derivative, small molecular carboxymethylcellulose, small molecular alginate, small molecular carbohydrate, small molecular uric acid, small molecular collagen, small molecular vegetative protein, small molecular gelatin, small molecular polylactate, small molecular polyethylene pyrrole, small molecular polyethylene glycol, peptide and thereof.

Preferably, said structure-linkage holes are round.

Preferably, this multi-dimensional flexible hydrophilic structure-linkage composite is prepared from materials choose from the combination of one of the groups of polyvinyl-chloride, polyethylene, ethylene vinyl acetate copolymer, polyvinyl alcohol, polyurethane, polypropylene or polyethylene, Teflon (PTFE), polysiloxane, polylactate derivatives, water-soluble polymethacrylic acid derivatives, cellulose derivatives, chitosan derivatives, carbon fiber derivatives and thereof.

The present invention consists of features and a combination of parts hereinafter fully described and illustrated in the accompanying drawings, it being understood that various changes in the details may be made without departing from the scope of the invention or sacrificing any of the advantages of the present invention.

### BRIEF DESCRIPTION OF THE DRAWINGS

To further clarify various aspects of some embodiments of the present invention, a more particular description of the invention will be rendered by references to specific embodiments thereof, which are illustrated in the appended drawings. It is appreciated that these drawings depict only typical embodiments of the invention and are therefore not to be considered limiting of its scope. The invention will be described and explained with additional specificity and detail through the accompanying drawings in which:

FIG. 1 illustrates a perspective view of the hydrogel surgical dressing product with a multi-dimensional flexible hydrophilic structure-linkage composite.

FIG. 2 illustrates (A) a cross-sectional view of the wound dressing product in the clinical application and (B) a perspective view of a joint region in clinic treatment.

### DETAILED DESCRIPTION OF THE INVENTION

The present invention will now be illustrated by the following examples, which are illustrative of, but not restrictive of, the present invention.

### DESCRIPTION OF NOTATION

1 a hydrogel surgical dressing
10 a multi-dimensional flexible hydrophilic structure-linkage composite
13 a hydrophilic multi-dimensional flexible structure-linkage membrane
15 a structure-linkage hole
20 a hydrogel cushion layer
23 a hydrophilic structure-linkage fiber
25 a hydrogel micro ball
30 a hydrophilic microsphere
100 a multi-dimensional flexible protecting film
1000 wound
2000 a joint region

### Example 1

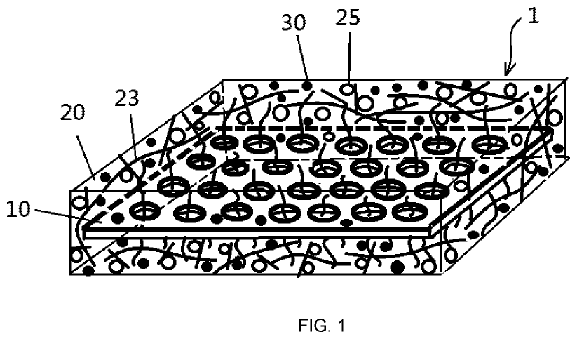

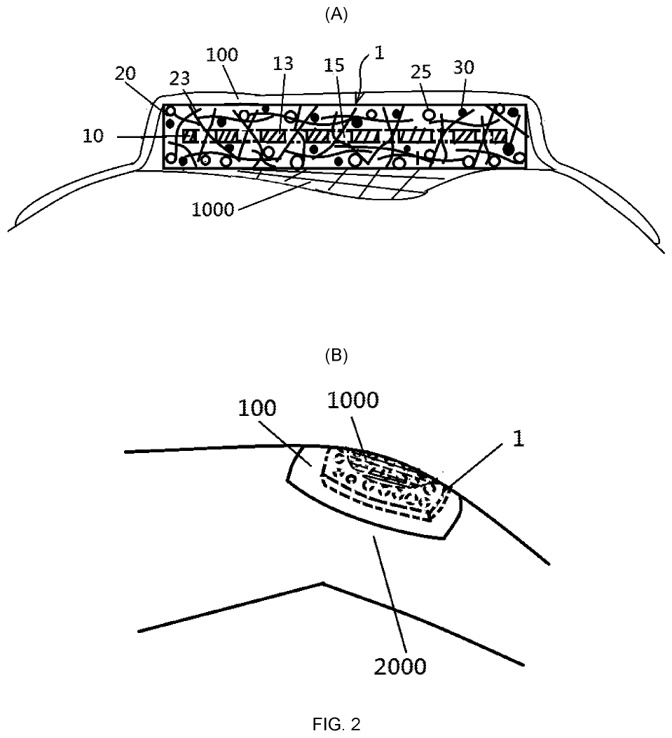

The present invention provides a hydrogel surgical dressing 1 having a multi-dimensional flexible hydrophilic structure-linkage composite 10, as shown in FIG. 1, comprising: a hydrogel cushion layer 20, and a multi-dimensional flexible hydrophilic structure-linkage composite, wherein said multi-dimensional flexible hydrophilic structure-linkage composite 10 comprising a hydrophilic multi-dimensional flexible structure-linkage membrane 13, and a multiple of structure-linkage holes 15 positioned into said hydrophilic multi-dimensional flexible structure-linkage membrane 13, and the sizes of structure-linkage holes 15 change with the degree of compelling force, resulting in a multi-dimensional flexible structure-linkage relationship. Said hydrogel cushion layer 20 comprising a multiple of hydrophilic structure-linkage fibers 23 and a multiple of hydrogel micro balls 25, said hydrophilic structure-linkage fiber 23 adsorbs side by side with said hydrogel micro ball in said hydrogel cushion layer 20; and said hydrogel cushion layer 20 is supported by a hydrophilic structure-linkage network forming through a network connection among said hydrophilic structure-linkage fibers 23 and said hydrophilic structure-linkage fibers 23, wherein said network connection comprising a first partial connection occurring into said structure-linkage holes 15, a second partial connection surrounding said structure-linkage holes 15, and a third connection linking said first partial connection and said second partial connection to form said multi-dimensional flexible structure-linkage relationship. Said hydrogel cushion layer comprises a multiple of hydrophilic microsphere 30 positioned in said hydrogel cushion layer 20, wherein said hydrophilic microsphere 30 adsorbs side by side with said hydrogel micro ball 25, and adsorbs side by side with said hydrophilic structure-linkage fiber 23, resulting in a consolidated structure of said hydrogel cushion layer 20. Because of the structure prepared from at least two kinds of polymeric network, the hydrophilic structure-linkage network preferably belongs to a kind of interpenetrating polymeric network or semi-interpenetrating polymer network. In the specific application, said hydrophilic microsphere 30 could be movably in said hydrophilic structure-linkage network. The mobility and efficiency of said hydrophilic microsphere 30 could be controlled by the size of said hydrophilic structure-linkage network. Preferably, said hydrophilic structure-linkage fiber 23 comprising a material selected from a group of polymethacrylate, polymethacrylate derivative, polysiloxane derivative, carboxymethylcellulose, alginate, polycarbohydrate, hyaluronic acid, collagen, vegetative protein, gelatin, polylactides, polyethylene pyrrole, polyethylene glycol, and thereof. Preferably, said hydrogel micro ball 25 comprising a material selected from a group of polymethacrylate, polymethacrylate derivative, polysiloxane derivative, carboxymethylcellulose, alginate, polycarbohydrate, hyaluronic acid, collagen, vegetative protein, gelatin, polylactides, polyethylene pyrrole, polyethylene glycol, and thereof. Preferably, said hydrophilic microspherel 30 comprising a material selected from a group of amino acid, water, oxygen, ethyl alcohol, propyl alcohol, glycerine, small molecular water-soluble polymethyl acrylic acid derivative, small molecular polysiloxane derivative, small molecular carboxymethylcellulose, small molecular alginate, small member carbohydrate, small molecular Bolivian uric acid, small molecular collagen, small molecular vegetative protein, small molecular gelatin, small molecular polylactate, small molecular polyethylene pyrrole, small molecular polyethylene glycol, peptide and thereof. Preferably, said structure-linkage holes are round. Preferably, this multi-dimensional flexible hydrophilic structure-linkage composite 10 was prepared from materials chosen from the combination of one of the groups of polyvinyl-chloride, polyethylene, ethylene vinyl acetate copolymer, polyvinyl alcohol, polyurethane, polypropylene or polyethylene, Teflon (PTFE), polysiloxane, polylactate derivatives, water-soluble polymethacrylic acid derivatives, cellulose derivatives, chitosan derivatives, carbon fiber derivatives and thereof. Particularly, the hydrogel surgical dressing 1 having a multi-dimensional flexible hydrophilic structure-linkage composite 10 is employed in clinical applications (see FIG. 2), the hydrogel surgical dressing 1 would contact with a target wound 1000. A multiple dimension flexible protecting film would be employed with said hydrogel surgical dressing 1 to fix said hydrogel surgical dressing 1 to cover said target wound 1000. At the same time, said multiple dimension flexible protecting film has to avoid contact with said target wound 1000. Said hydrophilic microsphere 30 could be movable in said hydrophilic structure-linkage network and employed to carry a nutrient, an enzyme, a growth factor, tissue, metabolite, etc. The mobility and carrying efficiency of said hydrophilic microsphere 30 could be controlled by the size of said hydrophilic structure-linkage network. The present invention provides a designed multi-dimensional flexible hydrophilic structure-linkage module in a hydrogel surgical dressing product to solve the clinical problems of traditional hydrogel dressings, in particular, falling off situation happening after swelling with water or tissue fluid in medical applications. The designed multi-dimensional flexible hydrophilic structure-linkage module provides a multi-dimensional flexible structure-linkage relationship of each parts of said hydrogel surgical dressing 10 instead of the traditional design of multiple adhering layers. The force inducing falling off situation could be dispersing into the multi-dimensional flexible hydrophilic structure-linkage composite. For example, medical treatments of a joint region 2000 was carried out.

Having described the invention in detail and by reference to preferred embodiments thereof, it will be apparent that modifications and variations are possible without departing from the scope of the invention which is defined in the appended claims.

1. A hydrogel surgical dressing product, comprising:
a hydrogel cushion layer; and
a multi-dimensional flexible hydrophilic structure-linkage composite, wherein
said multi-dimensional flexible hydrophilic structure-linkage composite comprising a hydrophilic multi-dimensional flexible structure-linkage membrane, and a multiple of structure-linkage holes positioned into said hydrophilic multi-dimensional flexible structure-linkage membrane, and the sizes of structure-linkage holes change with the degree of compelling force, resulting in a multi-dimensional flexible structure-linkage relationship; characterized in that
said hydrogel cushion layer comprising a multiple of hydrophilic structure-linkage fibers and a multiple of hydrogel micro balls, said hydrophilic structure-linkage fiber adsorbs side by side with said hydrogel micro ball in said hydrogel cushion layer; and said hydrogel cushion layer is supported by a hydrophilic structure-linkage network forming through a network connection among said hydrophilic structure-linkage fibers and said hydrophilic structure-linkage fibers, wherein said network connection comprising a first partial connection occurring into said structure-linkage holes, a second partial connection surrounding said structure-linkage holes, and a third connection linking said first partial connection and said second partial connection to form said structure-linkage multi-dimensional flexible structure-linkage relationship; and
a multiple of hydrophilic microsphere is positioned in said hydrogel cushion layer, wherein said hydrophilic microsphere adsorbs side by side with said hydrogel micro ball, and adsorbs side by side with said hydrophilic structure-linkage fiber, resulting in a consolidated structure of said hydrogel cushion layer.

2. A hydrogel surgical dressing product as claimed in claim 1, wherein said hydrophilic structure-linkage fiber comprising a material selected from a group of polymethacrylate, polymethacrylate derivative, polysiloxane derivative, carboxymethylcellulose, alginate, polycarbohydrate, hyaluronic acid, collagen, vegetative protein, gelatin, polylactides, polyethylene pyrrole, polyethylene glycol, and thereof.

3. A hydrogel surgical dressing product as claimed in claim 1, wherein said hydrogel micro ball comprising a material selected from a group of polymethacrylate, polymethacrylate derivative, polysiloxane derivative, carboxymethylcellulose, alginate, polycarbohydrate, hyaluronic acid, collagen, the vegetative protein, gelatin, polylactides, polyethylene pyrrole, polyethylene glycol, and thereof.

4. A hydrogel surgical dressing product as claimed in claim 1, wherein said hydrophilic microsphere comprising a material selected from a group of amino acid, water, oxygen, ethyl alcohol, propyl alcohol, glycerine, small molecular water-soluble polymethyl acrylic acid derivative, small molecular polysiloxane derivative, small molecular carboxymethylcellulose, small molecular alginate, small member carbohydrate, small molecular uric acid, small molecular collagen, small molecular vegetative protein, small molecular gelatin, small molecular polylactate, small molecular polyethylene pyrrole, small molecular polyethylene glycol, peptide and thereof.

5. A hydrogel surgical dressing product as claimed in claim 1, wherein said multi-dimensional flexible hydrophilic structure-linkage composite prepared from materials chosen from the combination of one of the groups of polyvinyl-chloride, polyethylene, ethylene vinyl acetate copolymer, polyvinyl alcohol, polyurethane, polypropylene or polyethylene, Teflon, polysiloxane, polylactate derivatives, water-soluble polymethacrylic acid derivatives, cellulose derivatives, chitosan derivatives, carbon fiber derivatives and thereof.

6. A hydrogel surgical dressing product as claimed in claim 1, wherein said structure-linkage holes are round.
